(19) United States
(12) Patent Application Publication
(10) Pub. No.: US 2011/0038814 A1
(43) Pub. Date: Feb. 17, 2011

(54) TRYPTASE INHIBITING MUSTARD EXTRACT

(76) Inventor: Emilie Rogg-Le Claire, Therwil (CH)

Correspondence Address: BIRCH STEWART KOLASCH & BIRCH, PO BOX 747, FALLS CHURCH, VA, 22040-0747, US

(21) Appl. No.: 12/674,333

(22) PCT Filed: Aug. 27, 2007
(86) PCT No.: PCT/EP07/07487

§ 371 Date: Feb. 19, 2010

(51) Int. Cl. A61K 36/31; A61K 8/97; A61P 17/00; A61Q 17/04

(52) U.S. Cl. 424/59; 424/755

(57) ABSTRACT

The present invention relates to mustard extracts (Brassicaceae), in particular extracts of the Sinapis alba L. type and/or of the Sinapis arvensis L. type, and to active concentrates containing same and having potent tryptase inhibitory activity. These can be used for topical skin and scalp care applications in form of creams, lotions, gels, shampoos and the like, for the cosmetic treatment of the epidermal barrier. Barrier abnormalities and disruptions respectively are often the starting point of itching and inflammatory skin diseases.

### BACKGROUND OF THE INVENTION

The role of the skin consists in insulating and mediating functions towards the environment. Various biochemical and biophysical systems help to maintain the integrity of this exposed organ, whereby the moisture content of the corneocytes and the lipids of the epidermal barrier exert a decisive role. The stratum corneum, the outermost layer of the skin prevents the skin from transepidermal water loss (TEWL). This permeability barrier is composed of lipids and can be damaged by many different influences, e.g. washing with tensides extracts lipids and thereby increases the water permeability of the stratum corneum. Moreover ultraviolet radiation catalyses a reticulation or disintegration of proteins, proteoglycans and polysaccharides, leading to a loss of elasticity and to an increased vulnerability of the stratum corneum. Exogeneous and endogeneous enzymes catalyse the degradation of proteins and other biomolecules in inflammatory processes and immune reactions. Tryptase, elastases and cathepsins attack skin proteins particularly leading to impaired barrier functions. Repeated barrier disruption induces epidermal hyperplasia and is thought to lead to dry skin. A weaken barrier shows enhanced TEWL and can result in xerotic skin conditions as well as in inflammatory dermatoses like atopic dermatitis (see Irvine et al., J. Invest. Dermatol. 126, 1200-2 (2006)), psoriasis, atopic eczema, and fibrosis. These dermatoses are characterized by a dry skin associated with pruritus (see Rawlings et al., J. Invest. Dermatol. 124, 1099-110 (2005); Yosipovitch, Int. J. Cosm. Science 26, 1-7 (2004); Gruber et al., J. Immunol. 158, 2310-7 (1997)). Most inflammatory skin lesions are covered with dry scales or scale-crusts. Even in the absence of recognizable scaly changes, xerotic skin changes such as atopic xerosis, associated with clinically invisible inflammation, display mild impairment in stratum corneum barrier function that is only demonstrable with biophysical measurement. The serine protease tryptase (E.C. no. 3.4.21.59) plays an important part in these processes. A strong correlation between tryptase activity and the TEWL level in the stratum corneum could be shown.

Tryptase is the most abundant serine protease in secretory granules of human mast cells, and is secreted from activated mast cells in parallel with histamine, heparin, cytokines and proteases, namely chymase and carboxypeptidase A. Mast cells are activated by different substances and mechanisms, such as IgE (immunoglobulin E), substance P (neuropeptide), C3a (protein fragment released from complement component C3) and C5a (protein fragment released from complement component C5), anaphylatoxins, TNF-α (tumor necrosis factor-α) and IL-1 (interleukin-1), free radicals, and UV radiation (see Schwartz, Clin. Allergy Immunol. 6, 9-23 (1995)). Moreover mast cell degranulation has been reported to occur in dry environmental conditions (see Ashida and Denda, Br. J. Dermatol. 149, 240-7 (2003)) and the activity of a tryptase-like enzyme in the stratum corneum was recently shown (see Voegeli et al., J. Invest. Dermatol. 126, 57 (2006)).

Tryptase has been shown to damage the dermal extracellular matrix (ECM) of the skin (see Banno, Res. Dev. Dep. 34, 46-52 (2006)). Moreover, tryptase induces a cascade of inflammatory responses. It has been shown to have a myriad of significant effects as a peptidase and protease that intensify the inflammatory response. Thus, for example tryptase is capable of degrading the basal membrane by splitting fibronectin and activating collagenase IV, uPA (urokinase) and MMP-3 (metalloproteinase-3) (see Hallgren and Pejler, FEBS Journal 273, 1871-95 (2006); Steinhoff et al., Arch. Dermatol. 139, 1479-88 (2003); Naukkarinen et al., J. Pathol. 180, 200-5 (1996)). Tryptase activates the Par-2 receptor (proteinase activated receptor-2) in sensory nerves, keratinocytes and mast cell membranes. Activation of the PAR-2 receptor in the sensory nerves induces the release of the neurotransmitter like neurokinin A, VIP (vaso intestinal peptide), SP (substance P), CGRP (calcitonin gene-related peptide) and somatostatin (see Steinhoff et al., J. Invest. Dermatol. 126, 1705-18 (2006)). These neurotransmitters are known to cause itching (see Yosipovitch, Cosm. Toil. 120, 55-8 (2005)). They also induce the proliferation of keratinocytes, a phenomenon known in inflammatory skin diseases (see Hsieh and Lin, J. Invest. Dermatol. 113, 579-86 (1999)). These neurotransmitters also allow a signal-amplifying by mast cell activation. The simultaneous keratinocyte activation by neurotransmitters and tryptase causes the synthesis and secretion of cytokine like IL-α or NGF neurotrophin (nerve growth factor) which induces the growth of nervous fibres and thus amplifies the signal (see Yosipovitch, Int. J. Cosm. Science 26, 1-7 (2004)).

Tryptase also intervenes in the inhibition of lipid synthesis and thus deregulates the functions of the stratum corneum (see Hachem et al., J. Invest. Dermatol. 126, 2074-86 (2006)), Tryptase also allows signal amplifying by mast cell activation. Consequently, tryptase is an enzyme exerting a central role in the first phase of the inflammatory reaction as well as in the chronic inflammation process. Therefore, the inhibition of this enzyme represents a potential target for the treatment of barrier abnormalities leading to xerotic skin conditions and inflammatory skin disorders, such as urticaria, eczematous dermatitis and hyperproliferative skin diseases like psoriasis.

Surprizingly it was found, that extracts of mustards (Brassicaceae), in particular of the Sinapis alba L. and/or of the Sinapis arvensis L. type, show a competitive tryptase-inhibiting activity and can be used for the topical treatment of skin and scalp barrier abnormalities. No cosmetic use of a Sinapis L. extract for tryptase inhibition has been published up to now. Tryptase inhibition for treating inflammatory diseases is known with synthetic compounds in the pharmaceutical field (see Burgess et al., U.S. Pat. No. 6,362,216).

### DESCRIPTION OF THE INVENTION

The present invention relates to extracts of mustards in particular of the Sinapis alba L. (Brassicaceae) type and/or of the Sinapis arvensis L, type, and to active fractions containing same and having potent tryptase-inhibiting activity. These can be used in topical formulations such as creams, lotions, gels, shampoos and the like, for the treatment especially of skin and/or scalp barrier abnormalities like xerotic skin conditions, (pruritus) itching, and/or dandruff and/or inflammatory skin disorders.

The mustard extract to be used in the present invention is preferably obtained from the seeds and/or from the aerial parts of the crop.

Finely ground, dried plant material, in particular of the Sinapis alba L. (Brassicaceae) and/or of the Sinapis arvensis L. type, may be extracted with different alcohols, such as ethanol, methanol or butanol, or with water the pH of which varies between 3 and 8, preferably from 4 to 6. Each of the cited solvents can be used alone or mixed. The used solvent volume can amount to 1 to 40 times, preferably 10 to 30 times the weight of plant material such as seeds to extract. For an efficient extraction, 10 to 30 times more solvent should be used. The extraction can be performed at 10 to 60° C., preferably at 25 to 50° C., for 1 to 48 hours, preferably for 1 to 30 hours. After filtration, the volume of the extract is reduced by distillation of the solvent to a great extent. The residue is diluted with a solution of alcohol, such as an aqueous ethanol 60% (v/v). The mixture is let settled for 12 to 24 hours at 4° C., whereupon it is filtered, concentrated and dried to obtain a dry extract. This extract tested for its tryptase-inhibiting activity has an IC₅₀ value of 0.64 mg/ml (dry extract).++

The topically effective extract of the present invention can be made available in any application form desired. Thus, these formulations can be, e.g., an aqueous or anhydrous preparation, an emulsion or micro-emulsion of the water-in-oil (w/o) or oil-in-water (o/w) type, a multiple emulsion, e.g., of the water-in-oil-in-water (w/o/w) type, a gel, a shampoo, a solid, or an aerosol. The formulations of the present invention may be available as, e.g., powder, wet patch, lotion, cream or ointment, shampoo and washing formulation, or in any other cosmetically approved form. The effective concentration of dry mustard extract is about 0.02 to 6%, preferably 0.05 to 3%, related to the total weight of the cosmetic composition. Stable active concentrates with a dry mustard extract content of 1 to 15%, preferably about 4 to 10%, related to the total weight of active concentrates, can be manufactured. These active concentrates may be formulated with glycerine or another compatible solvent, such as propylene glycol, or a mixture thereof. Optionally preservatives can be added.

The cosmetically and/or dermatologically effective extract of the present invention which can be made available in any application form desired, can be used together with any further, usually applied and topically applicable skin care agent. Examples of additional skin care agents are plant, animal or synthetic and semi-synthetic substances, such as moisturizing and barrier agents, skin-revitalizing agents, anti-itching or anti-dandruff ingredients, desquamation agents, antioxidants, UV ray-absorbing compounds and UV quenchers, vitamins, such as vitamin A, vitamin B, vitamin C and/or vitamin E, retinoids, peptides such as di-, tri-, tetra- and pentapeptides and derivatives thereof, hydroxy acids, anti-inflammatory agents, flavonoids, antimicrobial agents, antifungal agents, and mixtures thereof.

Acceptable carriers may generally be used for the manufacture of the cosmetically and/or dermatologically active composition or formulation of the present invention. Examples of such carriers are, alcohols, polyols, fatty acids, lipids, oils, waxes, thickeners, surfactants, emulsifiers, bulking agents, preservatives, aromas and fragrances as well as staining agents, foam stabilizers and/or silicones.

Carriers to be used in the present invention are in particular glycerine, polyglycerine compounds, ethylene glycol, propylene glycol, polyethylene glycols, polypropylene glycols, ethyl alcohol, isopropyl alcohol, agar gum, gum tragacanth, gum arabic, plant or animal gelatine, methyl cellulose, ethyl cellulose, carboxymethyl cellulose, hydroxymethyl cellulose, hydroxypropyl cellulose, sodium alginate, polyvinyl alcohol, polyvinyl alcohol acetate ester, C₆₋₂₂ fatty alcohols such as cetyl alcohol, C₆₋₂₂ fatty alcohol esters, in particular of stearic acid, palmitic acid, lauric acid and corresponding methyl, ethyl and propyl esters, lanolin, liquid paraffins or natural or synthetic waxes, such as Vaseline or beeswax, vegetal oils such as olive oil, coconut oil, soybean oil, castor oil and corresponding hardened oils, hydroxyl-containing compounds modified with polyalkylene oxides, as well as further raw materials known to be incorporated in dermatologically, cosmetic and/or therapeutic formulations.

For the preparation of a water-in-oil (w/o), oil-in-water (o/w) or water-in-oil-in-water (w/o/w) emulsion or microemulsion, compounds known per se and applied for this purpose are preferably used. For the preparation of the lipid phase, mineral or natural oils or waxes are preferably used. Synthetically manufactured esters of fatty acids with alcohols, such as esters of fatty acids with ethanol, propanol, isopropanol, propylene glycol or glycerine, or esters of fatty alcohols with organic C₃₋₂₀ acids, may be used too. E.g. esters of myristic acid, palmitic acid, stearic acid, oleic acid, such as propyl myristate, isopropyl palmitate, isopropyl stearate, isopropyl oleate, butyl stearate, hexyl laurate, 2-hexyldecyl stearate, or natural oils, such as jojoba oil, or a mixture thereof are preferred. Preferred silicones are in particular dimethyl polysiloxanes, preferably in cyclic or linear form.

Furthermore, the formulations of the present invention may comprise acids or bases for pH adjustment, e.g. sodium hydroxide, phosphoric acid, citric acid or lactic acid triethanolamine, preferably as a buffer system.

The following examples are intended to explain the present invention more specifically without limiting its scope in any manner.

### EXAMPLES

### Example 1

### Preparation of an Extract of Sinapis arvensis L.

Finely ground Sinapis alba L. seeds (100 g) from Dixa AG. (St. Gallen, Switzerland) are extracted with 900 g of water for 3 h at 40° C. After filtration on paper filter (Whatman), the obtained filtrate is concentrated on a rotary evaporator until obtaining 50 g of concentrate. After addition of 400 g of 96% ethanol (v/v), the mixture is let rest for 12 h at 4° C., whereupon it is filtered, concentrated and lyophilized to obtain 10.2 g of dry extract.

### Example 2

### Formulation of an Active Preparation with Sinapis alba L.

3 g of dry extract is formulated with 12 g of water, 35 g of glycerine, 0.15% of potassium sorbate and 0.3% of sodium benzoate, whereupon the pH is adjusted to 4.3 with citric acid. After filtration, the obtained concentrate shows the following characteristics: clear, brown coloured, slightly viscous liquid with a characteristic odour, a relative density (D_{20/20}) of 1.186 and a refractive index of 1.428.

### Example 3

### Tryptase Inhibitory Effect of an Extract of Sinapis alba L.

Enzymatic assays are performed at room temperature in 96-well microtiter plates. rhSkin beta tryptase (Promega, Madison, USA) is activated with a Tris-buffer comprising buffer 1 (50 mM Tris HCl, pH 7.6) and buffer 2 (50 mM Tris HCl, 1.65 M NaCl and 550 μg/ml of heparin, pH 7.6) in a 1:1 ratio. In standard incubations, 50 μl of tryptase (c=1.43 μg/ml) is added to the wells in a total volume of 125 μl of buffer 1. 25 μl of inhibitors is added and the mixture is incubated for 30 minutes. Thereafter, 25 μl of a 1 mM solution in water of the chromogenic peptide substrate Tos-Gly-Pro-Lys-pNA (Pentapharm Ltd., Basel, Switzerland) is added. The mustard extract is tested six times at five concentrations; the highest concentration tested being 2.8 mg/ml, Pefabloc® Tryp (1,4-Bis-[(Nα-2-naphthylsulfonyl)-3-amidino-(D,L)-phenylalanine]-piperazidedihydrochloride) (Pentapharm, Switzerland) is used as a positive control at a concentration of 37.8 nM.

The release of p-nitroaniline is recorded for 30 min at 405 nm using a photometer. The absorption differences per min (DA/min) are determined therefrom and the elastase inhibition is finally calculated in % using the following equation:

% inhibition=[(DA_{ref}/min−DAₛₐₘₚₗₑ/min)/(DA_{ref}/min)]×100

An IC₅₀ value of 0.64 mg/ml (related to the dry extract) could be determined for the mustard extract by means of a dose-response curve.

### Example 4

### Correlation of TEWL and Tryptase Activity in the Stratum Corneum

Twelve healthy Caucasian subjects (skin type II-III) participated in the study. All volunteers signed informed consent forms. Before conducting the sequential tape stripping (D-Squame®, CuDerm Corporation, Dallas, USA) on the cheek (9 times) TEWL was measured using an Aquaflux AF103 (Biox Systems, London, UK). The subjects were required not to apply any topical drugs or cosmetics for at least 12 hours before the stratum corneum was sampled. Firstly, 15 minutes before the tape stripping procedure, the skin was carefully cleaned with a cotton pad soaked with distilled water of ambient temperature and allowed to dry. The subjects were acclimated in an environmental room under standard conditions. The skin sites were marked with a surgical marker to ensure that the measurement probes and the tapes were consistently applied to the same area.

Standard D-Squame® disks with a diameter of 2.2 cm and an area of 3.8 cm² were placed on the skin under 225 g/cm² of pressure with a pressure device (CuDerm Corporation, Dallas, USA) for 5 seconds. The interval between the strippings was 20±5 seconds.

The protein content of the tape strippings was quantified by absorption measurements at 850 nm with the infrared densitometer SquameScan™ 850A (Heiland electronic, Wetzlar, Germany). SquameScan™ 850A is especially designed for the application of standard D-Squame® disks. For protein quantification the following equation was used:

C_{protein[μg/cm}²_{]}=1.366·Absorption[%]−1.557

Immediately after absorption measurement each tape stripping was transferred into a 1.5 ml Eppendorf tube and extracted for 15 min at 25° C. and 1000 rpm in 750 μl of a buffer composed of 0.1M Tris/HCl and 0.5% Triton X-100 at pH 8.0. The extracts of tape strippings were pooled. To 250 μl of the solutions 1.25 μl of 5 mM fluorogenic tryptase substrate Tos-Gly-Pro-Lys-AMC (Bachem, Switzerland) dissolved in DMSO were added (final substrate concentration=25 μM). The solutions were mixed at 37° C. and 1000 rpm. The reaction was stopped after 2 hours by adding 100 μl of acetic acid 1% to 100 μl of reaction mixture. The released AMC was quantified by a C18 HPLC gradient elution (80% water/20% acetonitrile/0.07% TFA to 50% water/50% acetonitrile/0.07% TFA). The column used was Symmetry C18, 3.5 μm, 4.6 mm×75 mm (Waters, Milford, USA). The flow rate was 1 ml/min, the injection volume 5 μl and the retention time of AMC 3.5 minutes. The wavelength for emission was 442 nm and for excitation 354 nm.

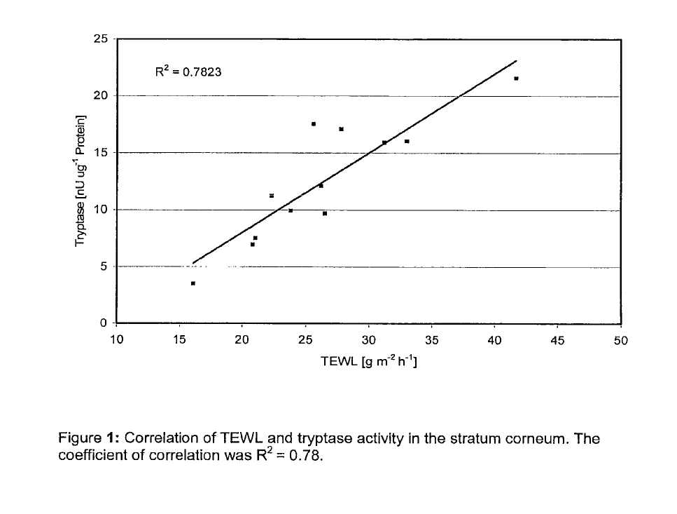

The data are summarized in Table 1 and FIG. 1

TABLE 1
Determination of the TEWL and tryptase level in the stratum
corneum of 12 subjects.

| | TEWL | Tryptase |
|---|---|---|
| Subject | [g m⁻² h⁻¹] | [nU μg⁻¹ protein] |
| 01 | 27.8 | 5.70 |
| 02 | 41.7 | 7.21 |
| 03 | 31.2 | 5.31 |
| 04 | 26.2 | 4.03 |
| 05 | 33.0 | 5.35 |
| 06 | 16.1 | 1.17 |
| 07 | 26.5 | 3.23 |
| 08 | 23.8 | 3.31 |
| 09 | 21.0 | 2.51 |
| 10 | 20.8 | 2.33 |
| 11 | 22.3 | 3.74 |
| 12 | 25.6 | 5.85 |

### Example 5

### Preparation of a Cosmetic Lotion

| Phase | Ingredients | INCI Name | % Wt. |
|---|---|---|---|
| A | Amphisol K | Potassium Cetyl Phosphate | 2.00 |
| | Tegosoft CT | Caprylic/Capric Triglyceride | 6.50 |
| | Tegosoft TN | C12-15 Alkyl Benzoate | 6.00 |
| | Tegin Pellets | Glyceryl Stearate | 3.00 |
| | Cetyl Alcohol | Cetyl Alcohol | 1.00 |
| B | Water | Water | 72.55 |
| | Glycerin | Glycerin | 3.00 |
| | Phenonip | Phenoxyethanol, Methylparaben, | 0.70 |
| | | Ethylparaben, Butylparaben, | |
| | | Propylparaben, Isobutylparaben | |
| C | Keltrol RD | Xanthan Gum | 0.25 |
| D | Citric Acid | Citric Acid | q.s. |
| E | Dry extract example 1 | | 2.00 |
| | | Total | 100.00 |

### Procedure

Disperse phase C in phase B. Heat separately phases A and BC. Add phase A to phase BC under stirring, then homogenise. Adjust the pH to approx. 4.5 with phase D. Add phase E under stirring. Control the pH and adjust if necessary to 4.5.

### Example 6

### Preparation of a Cosmetic Gel

| Phase | Ingredients | INCI Name | % Wt. |
|---|---|---|---|
| A | Water | Water | 90.30 |
| | Natrosol 250 HHR | Hydroxyethylcellulose | 1.00 |
| B | Glycerin | Glycerin | 3.00 |
| | Phenonip | Phenoxyethanol, Methylparaben, | 0.70 |
| | | Ethylparaben, Butylparaben, | |
| | | Propylparaben, Isobutylparaben | |
| C | NaOH 30% | Sodium Hydroxide | q.s. |
| D | Citric Acid 50% | Citric Acid | q.s. |
| E | Preparation example 2 | | 5.00 |
| | | Total | 100.00 |

### Procedure

Disperse the Natrosol 250 HHR in water. Mix phase B, then add it to phase A. Adjust the pH to approx. 8.5 with phase C to obtain a clear gel. Then adjust the pH to approx. 4.5 with phase D (the gel gets opalescent). Add phase E under stirring. Control the pH and adjust if necessary to 4.5.

1. A mustard extract obtained from plants of the brassicaceae family.

2. A mustard extract according to claim 1 obtained from sinapis alba L. type and/or sinapis arvensis L. type plants.

3. A mustard extract according to claim 1 or 2 thereby characterized that it is obtained from the seeds and/or from the aerial parts of the crop.

4. Use of a mustard extract according to claim 1 for non therapeutic cosmetic topical application, in particular for the treatment of skin and/or scalp barrier abnormalities like xerotic skin conditions, itching, and/or dandruff and/or inflammatory skin disorders.

5. Use of a mustard extract according to claim 1 for the preparation of therapeutically and/or cosmetically active compositions, in particular as skin and/or scalp care products.

6. Use of a mustard extract according to claim 5 thereby characterized that the composition is encapsulated in nanoparticles such as liposomes, nanosomes, cyclodextrins.

7. A skin and/or scalp care composition containing a mustard extract according to claim 1.

8. A skin and/or scalp care composition containing a dry mustard extract from sinapis alba L and/or sinapis arvensis L.

9. A skin and/or scalp care composition containing a dry mustard extract as defined in claim 8 in a quantity ranging between 0.02-6% (m/m), preferably between 0.05-3% (m/m).

10. A skin and/or scalp care composition containing an active concentrate made with a dry mustard extract from sinapis alba L and/or sinapis arvensis L.

11. A skin and/or scalp care composition containing an active concentrate made with a dry mustard extract as defined in claim 10 in a quantity ranging between 1-15% (m/m), preferably between 4-10% (m/m).

12. A skin and/or scalp care composition thereby characterized that it contains a) a mustard extract according to claim 1, and b) a safe and efficient quantity of at least one additional skin and/or scalp care agent selected from the group comprising plant, animal or synthetic and semi-synthetic substances, such as moisturizing and barrier agents, skin-revitalizing agents, anti-itching ingredients, desquamation agents, antioxidants, UV ray-absorbing compounds and UV quenchers, vitamins, such as vitamin A, vitamin B, vitamin C and/or vitamin E, retinoids, peptides such as di-, tri-, tetra- and pentapeptides and derivatives thereof, hydroxy acids, anti-inflammatory agents, flavonoids, antimicrobial agents, antifungal agents, and mixtures thereof.

13. A skin and/or scalp care composition according to claim 7 thereby characterized that it comprises one or more topically acceptable carriers.

14. A composition according to claim 7, thereby characterized that it can be in the form of an anhydrous preparation, an emulsion or micro-emulsion of the water-in-oil (w/o) or oil-in-water (o/w) type, a multiple emulsion, e.g., of the water-in-oil-in-water (w/o/w) type, a gel, a solid, an ointment, a shampoo, a washing formulation, an aerosol or in any other cosmetically or therapeutically approved form.

15. Use of a composition according to claim 7 as a non-therapeutic cosmetically active product, in particular for the treatment of skin and/or scalp barrier abnormalities like xerotic skin conditions, itching, and/or dandruff and/or inflammatory skin disorders.

16. A method for the non-therapeutic cosmetic treatment of skin and/or scalp barrier abnormalities like xerotic skin conditions, itching and/or dandruff and/or inflammatory skin disorders, thereby characterized that a composition containing a mustard extract according to claim 7 is applied onto the skin and/or the scalp.

17. A method for the therapeutic cosmetic treatment of skin and/or scalp barrier abnormalities like xerotic skin conditions, itching and/or dandruff and/or inflammatory skin disorders, thereby characterized that a composition containing a mustard extract according to claim 7 is applied onto the skin and/or the scalp.
